(19) United States
(12) Patent Application Publication
(10) Pub. No.: US 2026/0232938 A1
(43) Pub. Date: Aug. 13, 2026

(54) NEONATAL AND PEDIATRIC NASAL CANNULAS

(71) Applicant: Nspire Co., Chicago, IL (US)

(72) Inventors: Liam Hawry, Chicago, IL (US); William James Fienup, Chicago, IL (US); Nickolaus Anton Stagl, Chicago, IL (US); Wadsworth Williams, Chicago, IL (US); Robert F. Rioux, Ashland, MA (US)

(21) Appl. No.: 19/534,201

(22) Filed: Feb. 9, 2026

Related U.S. Application Data
(60) Provisional application No. 63/755,580, filed on Feb. 7, 2025.

(51) Int. Cl. A61M 16/06; A61M 16/20

(52) U.S. Cl. CPC A61M 16/0666; A61M 16/0688; A61M 16/208; A61M 2240/00

(57) ABSTRACT

The present invention is a nasal cannula that allows a pediatric patient to both inhale and exhale by using only his or her nose, while simultaneously using his or her mouth for something else, such as breast-or bottle-feeding through his or her mouth. The cannula also fits and conforms into the nostrils of the pediatric patient, thereby allowing the pediatric patient to inhale and exhale only through his or her nose while still generating positive end-expiratory pressure (PEEP).

### CROSS-REFERENCE TO RELATED APPLICATIONS

This application claims priority to, and the benefit of, U.S. Provisional Application No. 63/755,580, filed on Feb. 7, 2025, the content of which is incorporated by reference herein in its entirety.

### TECHNICAL FIELD

The present disclosure relates generally to neonatal and pediatric nasal cannulas, and, more particularly, to an improved nasal cannula design allowing a pediatric patient to both inhale and exhale using only their nose, while still generating positive end-expiratory pressure (PEEP), and further simultaneously allowing the patient to use their mouth for a different activity (e.g., breast-or bottle-feeding through their mouth).

### BACKGROUND

A nasal cannula is a device through which supplemental oxygen is delivered into one or both nostrils of a patient. The device is connected to supply tubing, and the supply tubing is connected to a source of oxygen. The extra oxygen that is delivered out of the source and into the supplying tubing (that typically is several feet in length, such as 7 feet or 14 feet long, for example) travels through the supply tubing until the extra oxygen reaches the device, and the supplemental oxygen then travels through the device and into the patient's nostril(s). In this way, the supplemental oxygen is delivered into the patient's body. Nasal cannulas made for newborn children (neonatal) or for children in general (pediatrics, including children from birth through adolescence, typically up to age 18-21 years old) are typically smaller than nasal cannulas made for adult humans or for other adult mammals.

One failing or shortcoming of existing nasal cannulas designed for children (particularly newborns or infants) is oxygen leak. With existing neonatal and pediatric nasal cannulas, the cannula can fall out of place on the child patient and/or it can be misaligned in its placement on the child patient, with the result in either case being that the proper amount of supplemental oxygen does not make it into the child's body. Existing neonatal and pediatric nasal cannulas thus make it difficult for treating physicians to titrate the correct amount of oxygen for fragile neonates. Too little oxygen can result in developmental delays or even death of the child patient, and too much oxygen can result in chronic lung disease and blindness in the child patient.

### SUMMARY

The invention of the present disclosure addresses the drawbacks and limitations of current nasal cannulas (designed specifically for children) by providing a nasal cannula having improvements in design and function.

In particular, when placed on a child patient, a neonatal or pediatric nasal cannula according to the invention is designed to allow the child patient to not only receive the supplied supplemental oxygen through the cannula and into his or her nostrils but also to exhale out of his or her nostrils through the cannula. The child patient thus can use his or her mouth to breast feed or bottle feed while simultaneously breathing in and out only through his or her nose and through the cannula.

A neonatal or pediatric nasal cannula according to the invention also is designed to allow the child patient to inhale and exhale only through his or her nose while still generating positive end-expiratory pressure (PEEP). The inventive cannula fits and conforms into the nostrils of the child patient. Both the shape of and the material(s) used to make the inventive cannula allow the cannula to adapt to the inner shape and volume of the child patient's nostrils. By occupying the normal shape of the nostril, the inventive cannula generates a certain (typically small) amount of pressure as air is exhaled by the child patient, and this results in keeping the child patient's smaller-than-adult airways open. Most nasal cannula, whether for children or adults, do not and cannot provide this added pressure feature because they do not create a seal. Generating PEEP is typically reserved for more invasive and expensive methods of supplemental oxygen therapy.

These and other aspects and details of cannulas according to the invention will become clearer by referring to the following parts of and/or attachments to this document, and various methods of making and using such cannulas will become clearer as well. The entirety of this document illustrates cannulas and methods that embody the invention, but the invention is not limited only to the specific cannulas and methods disclosed herein. Various ideas and combinations that can be derived from the contents of this document and/or attachments to it (including both text and accompanying drawings) are to be considered as disclosed and included herein.

### BRIEF DESCRIPTION OF THE DRAWINGS

Features and advantages of the claimed subject matter will be apparent from the following detailed description of embodiments consistent therewith, which description should be considered with reference to the accompanying drawings.

FIG. 1 is a perspective view showing an embodiment of a nasal cannula consistent with the present disclosure placed upon a face of a pediatric patient (i.e., infant), illustrating positive attributes of a mask maintaining airflow while not obstructing the infant's mouth, thereby enabling the infant to feed.

FIGS. 2 and 3 is a perspective front and rear views, respectively, of a nasal cannula consistent with the present disclosure.

FIGS. 4 and 5 are front and rear views, respectively, of the nasal cannula.

FIGS. 6 and 7 are top and bottom views, respectively, of the nasal cannula.

FIGS. 8 and 9 are a perspective views of the nasal cannula with the valve housing member removed to better illustrate the interior of the central hub and airflow passageways therein.

FIG. 10 is an enlarged perspective view, partly in section, of the nasal cannula with the valve housing removed and showing the inflow and outflow passageways of a nostril insertion member to better illustrate incoming airflow (for inhalation) and outgoing airflow (e.g., exhalation).

FIG. 11 is an enlarged perspective view, partly in section, of the nasal cannula with the valve housing in place and showing the inflow and outflow passageways of a nostril insertion member to better illustrate incoming airflow (for inhalation) and outgoing airflow (e.g., exhalation).

FIG. 12 is an enlarged perspective view, partly in section, of the nasal cannula with the valve housing in place and including associated one-way valves to allow for outgoing airflow (e.g., exhalation) to pass.

FIG. 13 is a sectional view of the nasal cannula with the valve housing in place and including associated one-way valves to allow for outgoing airflow (e.g., exhalation) to pass.

FIGS. 14A, 14B, and 14C are side views illustrating an alternative embodiment of a cheek pad consistent with the present disclosure and allowing for temporary device removal from the cheek pad.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present disclosure relates generally to neonatal and pediatric nasal cannulas. In particular, the present invention is a nasal cannula that allows a pediatric patient to both inhale and exhale by using only his or her nose, while simultaneously using his or her mouth for something else, such as breast-or bottle-feeding through his or her mouth. The cannula also fits and conforms into the nostrils of the pediatric patient, thereby allowing the pediatric patient to inhale and exhale only through his or her nose while still generating positive end-expiratory pressure (PEEP).

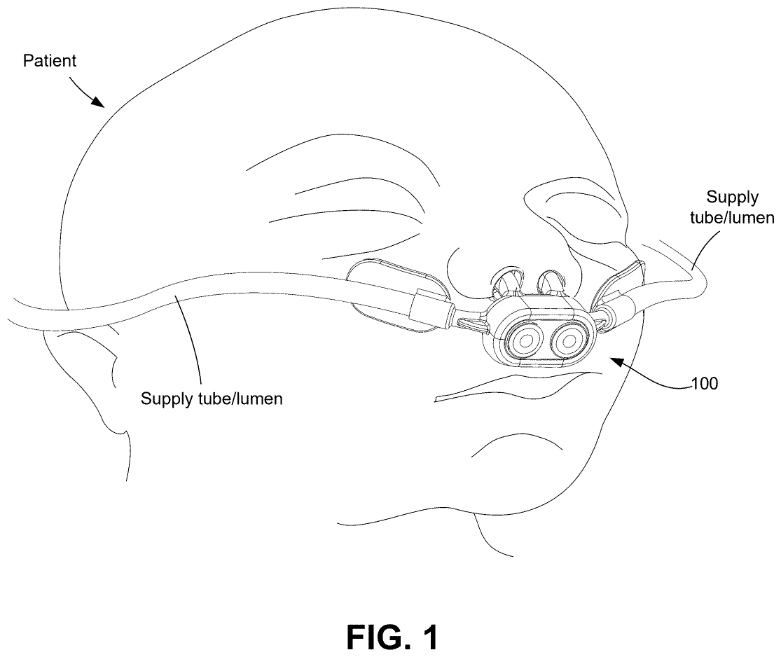

FIG. 1 is a perspective view showing an embodiment of a nasal cannula 100 consistent with the present disclosure placed upon a face of a pediatric patient (i.e., infant, toddler, child, adolescent, etc.), illustrating positive attributes of a mask maintaining airflow while not obstructing the infant's mouth, thereby enabling the infant to feed.

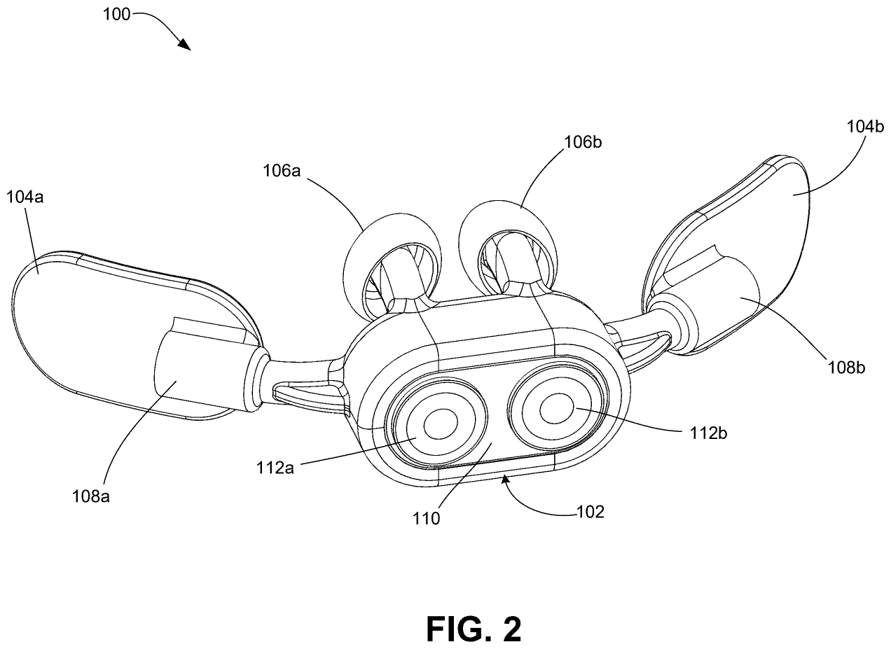

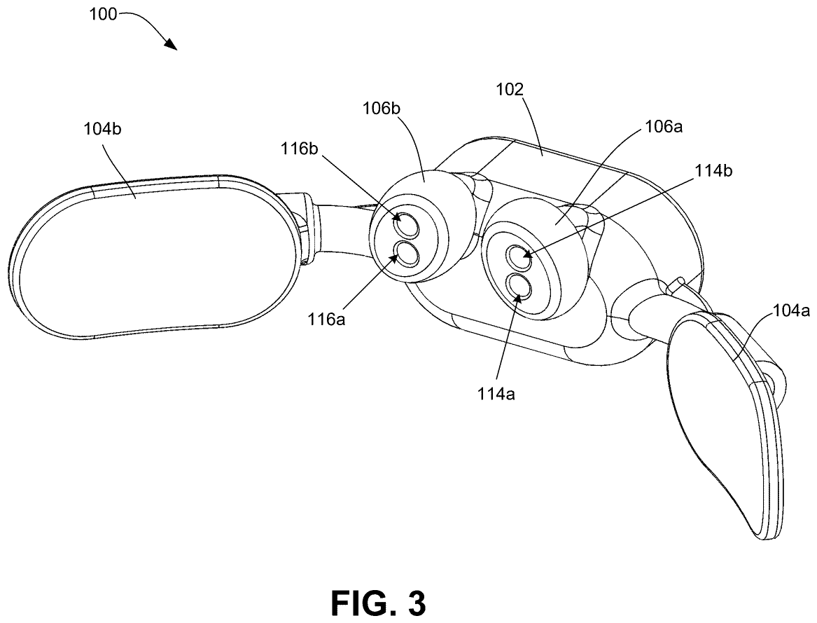

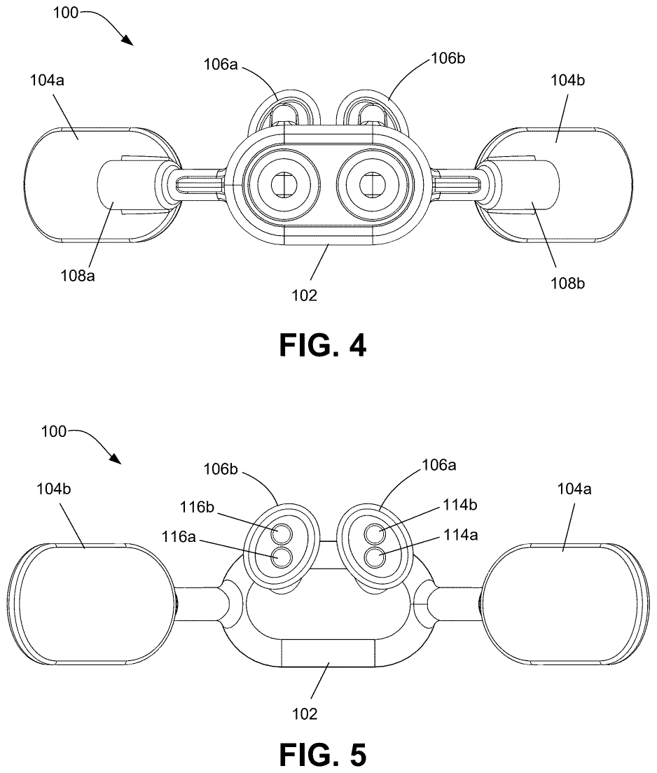

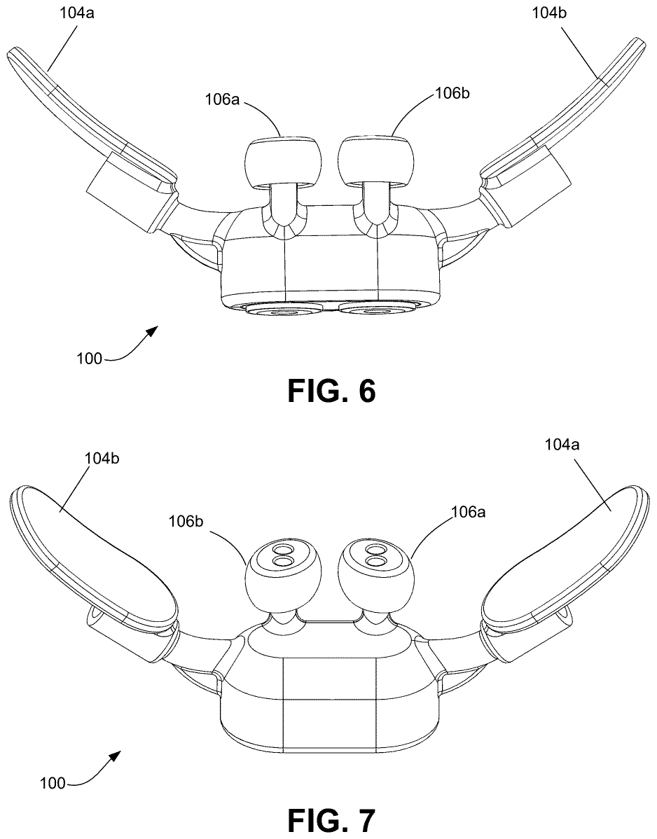

FIGS. 2 and 3 is a perspective front and rear views, respectively, of a nasal cannula 100 consistent with the present disclosure. FIGS. 4 and 5 are front and rear views, respectively, of the nasal cannula 100. FIGS. 6 and 7 are top and bottom views, respectively, of the nasal cannula 100.

As shown, the nasal cannula 100 includes a central hub portion 102, a first cheek pad 104a extending from one side of the central hub 102 and for contacting a right cheek of the pediatric patient, and a second cheek pad 104b extending from an opposite side of the central hub 102 and for contacting a left cheek of the pediatric patient. The cannula 100 further includes a pair of nostril insertion members 106a, 106b (also referred to herein as “nasal caps”) extending from the central hub 102. A first nostril insertion member 106a is intended for insertion into a right nostril of the pediatric patient and a second nostril insertion member 106b is intended for insertion into a left nostril of the pediatric patient. The cannula 100 further includes a pair of connector inhale ports 108a, 108b, each for attaching to an end of a respective supply tube or lumen (which can be coupled to a gas supply delivery system for the delivery of gas, such as oxygen, to the patient). A first connector inhale port 108a is located at or in proximity to the first cheek pad 104a and a second connector inhale port 108b is located at or in proximity to the second cheek pad 104b. The cannula 100 further includes a valve housing 110 provided at the central hub portion 102. As will be described in greater detail herein, the valve housing 110 allows gas (e.g., oxygen) to pass from the supply tubes and through inflow passageways of each of the nasal caps 106a, 106b (for inhalation by the patient) and further allows exhalation by the patient to pass through outflow passageways of each of the nasal caps 106a, 106b (and out of the valve housing 110 by way of one-way valves 112a, 112b.

Referring to FIGS. 3 and 5, for example, each of the first and second nasal caps 106a, 106b includes a pair of lumens respectively allowing for inhalation and exhalation. In particular, the first nasal cap 106a includes an inflow passageway for allowing gas to pass into the patient's right nostril via an inhalation lumen 114a in fluid communication with the inflow passageway. The first nasal cap 106a also includes an outflow passageway for allowing exhalation by the patient to pass out of the patient's right nostril and into the outflow passageway via an exhalation lumen 114b. Similarly, the second nasal cap 106b includes an inflow passageway for allowing gas to pass into the patient's left nostril via an inhalation lumen 116a in fluid communication with the inflow passageway of the second nasal cap 106b. The second nasal cap 106b also includes an outflow passageway for allowing exhalation by the patient to pass out of the patient's left nostril and into the outflow passageway of the second nasal cap 106b via an exhalation lumen 116b. Accordingly, the unique design of first and second nasal caps 106a, 106b allows for a pediatric patient to not only receive the supplied supplemental oxygen through the cannula and into his or her nostrils but also to exhale out of his or her nostrils through the cannula. The child patient thus can use his or her mouth to breast feed or bottle feed while simultaneously breathing in and out only through his or her nose and through the cannula.

The nasal cannula 100 is further designed to allow the pediatric patient to inhale and exhale only through his or her nose while still generating positive end-expiratory pressure (PEEP). More specifically, the cannula is designed to provide an improved fit and conformation into the nostrils of the patient. For example, both the shape of and the material(s) used to make the inventive cannula allow the cannula to adapt to the inner shape and volume of the pediatric patient's nostrils. In particular, portions of the first and second nasal caps may generally be composed from a relatively thin and flexible material, allowing for each nasal cap to better conform to nasal lumen of the patient's nostrils. For example, the first and second nasal caps may generally be made from a lightweight, flexible, and/or kink/crush resistant material, such as a medical grade silicone and/or soft polyvinyl chloride material. Furthermore, the nasal caps, as well as other portions of the cannula, may be manufactured by any known technique, including, but not limited to, injection molding, dip molding, and the like.

By occupying the normal shape of the nostril, the inventive cannula generates a certain (typically small) amount of pressure as air is exhaled by the pediatric patient, and this results in keeping the pediatric patient's smaller-than-adult airways open. Existing nasal cannulas, whether for children or adults, do not and cannot provide this added pressure feature because they do not create a seal. Furthermore, the first and second cheek pads 104a, 104b may be shaped so as to better conform to a pediatric patient's cheeks. For example, referring to FIGS. 6 and 7, the first and second cheek pads 104a, 104b may have a curved shape, improving contact and conformation with the patient's normal shape of their face.

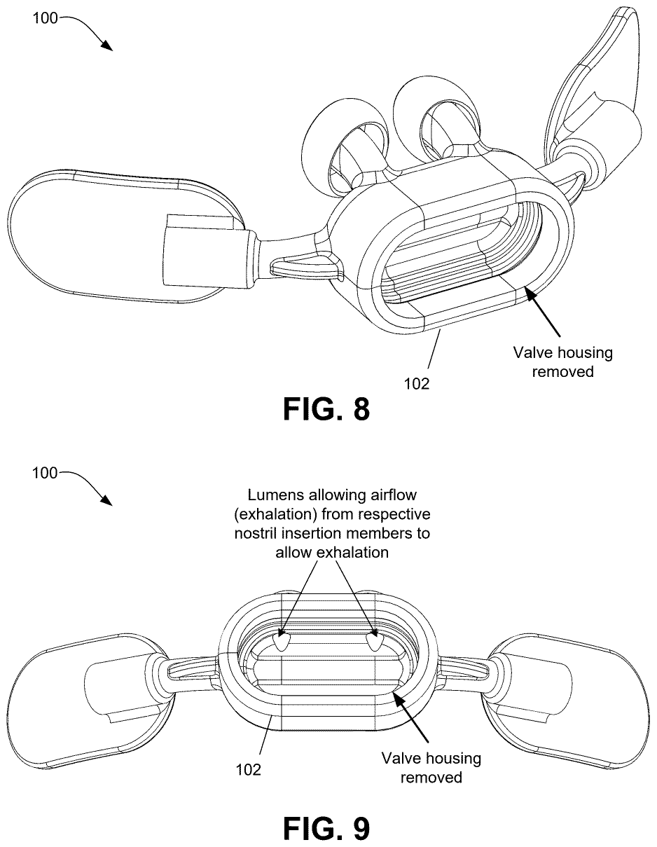

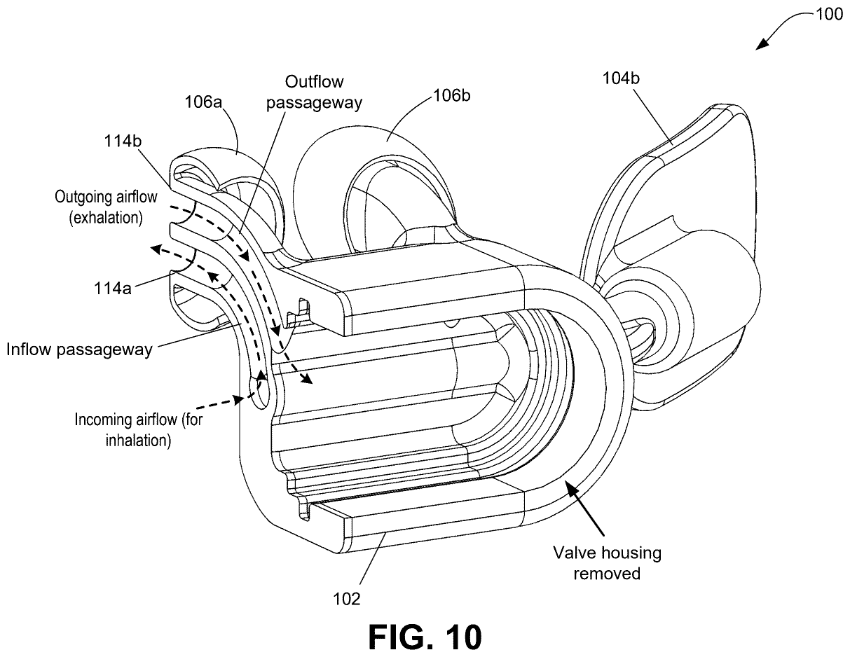

FIGS. 8 and 9 are a perspective views of the nasal cannula 100 with the valve housing member 110 removed to better illustrate the interior of the central hub 102 and airflow passageways therein. FIG. 10 is an enlarged perspective view, partly in section, of the nasal cannula 100 with the valve housing 110 removed and showing the inflow and outflow passageways of the first nasal cap 106a to better illustrate incoming airflow (for inhalation) and outgoing airflow (e.g., exhalation). As shown, the first nasal cap 106a has an inflow passageway for allowing gas (i.e., supplemental oxygen) to pass into the right nostril of the pediatric patient, via the inhalation lumen 114a. The inflow passageway is in fluid communication with the first connected inhale port 108a, such that the inflow passageway carries incoming airflow, including supplemental oxygen, from the supply tube. As shown, the inflow passageway includes multiple corners and radii, thereby enhancing hydration of the incoming airflow. The first nasal cap 106a includes an outflow passageway for allowing exhalation by the pediatric patient to pass out of the right nostril of the pediatric patient, via the exhalation lumen 114b, and out of the cannula, via an associated one-way valve 112a of the valve housing 110.

It should be noted that the second nasal cap 106b has a similar configuration as the first nasal cap 106a, in that the second nasal cap 106b includes an inflow passageway for allowing gas (i.e., supplemental oxygen) to pass into the left nostril of the pediatric patient, via the inhalation lumen 116a (the inflow passageway being in fluid communication with the second connected inhale port 108b, such that the inflow passageway carries incoming airflow, including supplemental oxygen, from the supply tube) and the second nasal cap 106b further includes an outflow passageway for allowing exhalation by the pediatric patient to pass out of the left nostril of the pediatric patient, via the exhalation lumen 116b, and out of the cannula, via an associated one-way valve 112b of the valve housing 110.

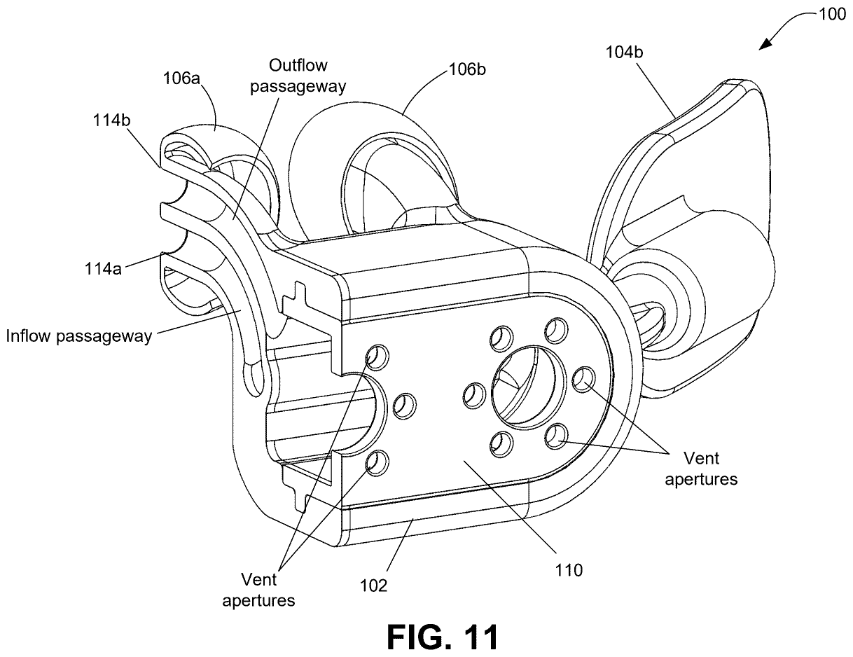

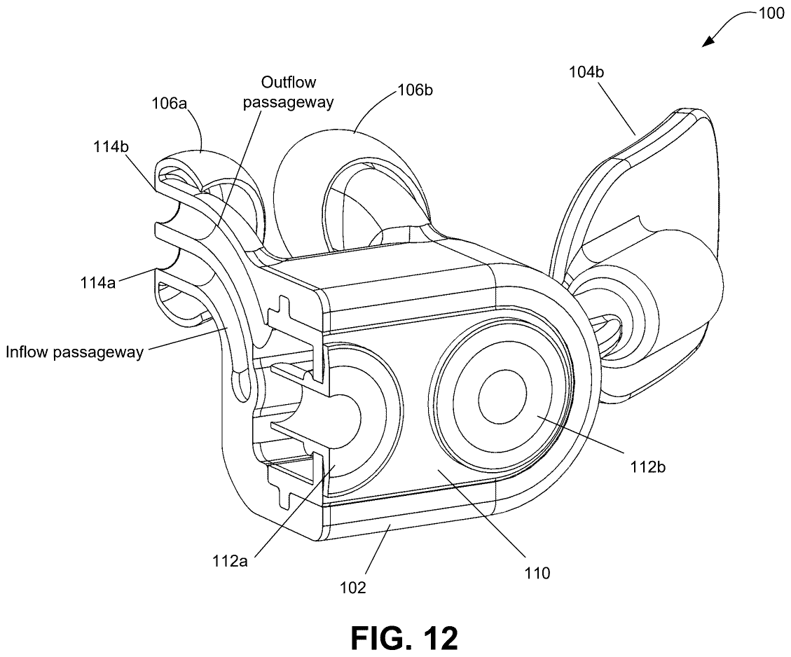

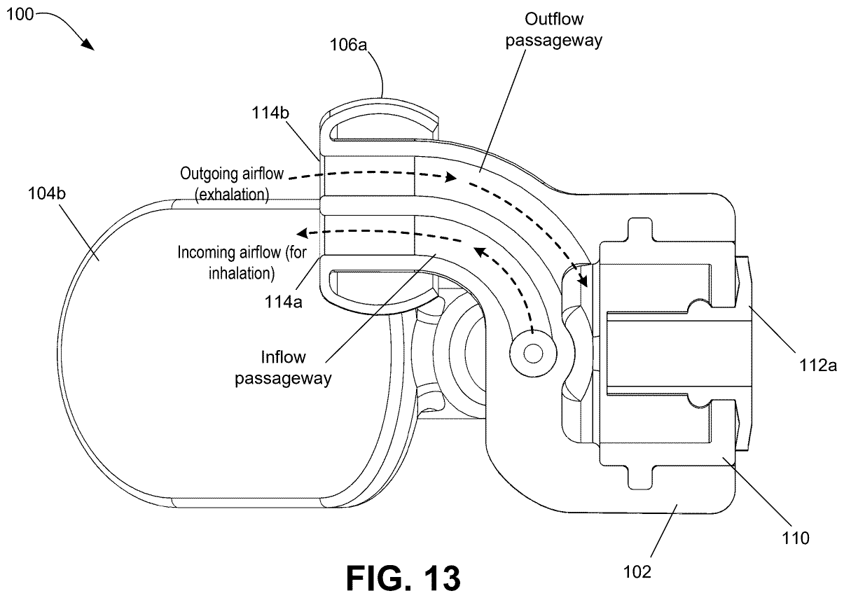

FIG. 11 is an enlarged perspective view, partly in section, of the nasal cannula 100 with the valve housing 110 in place and showing the inflow and outflow passageways of a nostril insertion member 106a to better illustrate incoming airflow (for inhalation) and outgoing airflow (e.g., exhalation). FIG. 12 is an enlarged perspective view, partly in section, and FIG. 13 is a cross-section view, of the nasal cannula with the valve housing in place and including associated one-way valves to allow for outgoing airflow (e.g., exhalation) to pass.

As shown, the valve housing 110 may be a molded material mechanically inserted into the central hub portion 102, and configured to hold one-way check valves 112a, 112b in place (via a mechanical coupling connection) for the respective nasal lumens (i.e., each outflow passageway of the respective first and second nasal caps 106a, 106b). As shown, the valve housing 110 may include a connection portion to which a respective valve 112a, 112b can be coupled. The connection portion may simply be a single aperture shaped and/or sized to receive and retain the respective valve 112a, 112b. Each connection portion further includes an array of vent apertures provided around the connection portion (i.e., radially positioned around a perimeter of the connection portion) to allow for outgoing airflow to pass (i.e., allowing for exhaled air to pass out of the valve housing). As shown, the valve housing 110 may include an umbrella valve feature on an anterior face of the valve housing 110, and a duckbill valve feature connected to the umbrella feature, thereby allowing for outgoing airflow to pass therethrough.

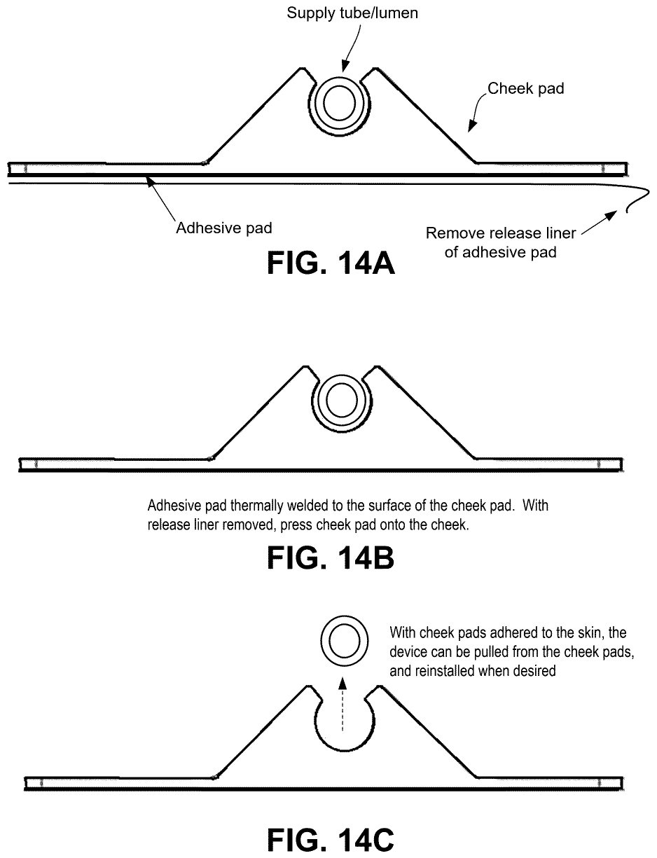

FIGS. 14A, 14B, and 14C are side views illustrating an alternative embodiment of a cheek pad consistent with the present disclosure and allowing for temporary device removal from the cheek pad. In this embodiment, the cheek pads 104a, 104b may include an adhesive pad thermally welded to a contact surface of the cheek pad (surface to contact the patient's cheek). Upon removing the release liner and exposing the adhesive pad, the cheek pad can simply be pressed against a patient's check to thereby adhere the cheek pad to the patient's cheek. Upon adhering the cheek pad(s) to the patient's skin, a supply tube or lumen can be disengaged from a respective cheek pad and reinstalled when desired, thereby providing a convenient manner in which the supply tube or lumen, or any device for that matter, can be temporarily removed from the cheek pads without requiring removal of the cannula itself.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A nasal cannula for use on a pediatric patient, comprising:
a central hub;
a first cheek pad extending from one side of the central hub and for contacting a right cheek of the pediatric patient;
a second cheek pad extending from an opposite side of the central hub and for contacting a left cheek of the pediatric patient;
a first nostril insertion member extending from the central hub and for insertion into a right nostril of the pediatric patient, the first nostril insertion member having an inflow passageway for allowing gas to pass into the right nostril of the pediatric patient, the first nostril insertion member also having an outflow passageway for allowing exhalation by the pediatric patient to pass out of the right nostril of the pediatric patient; and
a second nostril insertion member extending from the central hub and for insertion into a left nostril of the pediatric patient, the second nostril insertion member having an inflow passageway for allowing the gas to pass into the left nostril of the pediatric patient, the second nostril insertion member also having an outflow passageway for allowing the exhalation by the pediatric patient to pass out of the left nostril of the pediatric patient.

2. The nasal cannula of claim 1, further comprising a first connector inhale port for attaching to an end of a supply tube.

3. The nasal cannula of claim 2, wherein the first connector inhale port is located near the first cheek pad.

4. The nasal cannula of claim 2, further comprising a second connector inhale port for attaching to another end of the supply tube.

5. The nasal cannula of claim 4, wherein the second connector inhale port is located near the second cheek pad.

6. The nasal cannula of claim 1, wherein the central hub includes a valve housing.

7. The nasal cannula of claim 6, wherein the valve housing allows the gas to pass through the inflow passageways.

8. The nasal cannula of claim 6, wherein the valve housing allows the exhalation to pass through the outflow passageways.

9. A nasal cannula for use on a pediatric patient, comprising:
a central hub;
a first nostril insertion member extending from the central hub and for insertion into a right nostril of the pediatric patient, the first nostril insertion member having an inflow passageway for allowing gas to pass into the right nostril of the pediatric patient, the first nostril insertion member also having an outflow passageway for allowing exhalation by the pediatric patient to pass out of the right nostril of the pediatric patient; and
a second nostril insertion member extending from the central hub and for insertion into a left nostril of the pediatric patient, the second nostril insertion member having an inflow passageway for allowing the gas to pass into the left nostril of the pediatric patient, the second nostril insertion member also having an outflow passageway for allowing the exhalation by the pediatric patient to pass out of the left nostril of the pediatric patient.

10. The nasal cannula of claim 9, further comprising:
a first cheek pad for contacting a right cheek of the pediatric patient; and
a second cheek for contacting a left cheek of the pediatric patient.

11. The nasal cannula of claim 10, further comprising a first connector inhale port for attaching to an end of a supply tube.

12. The nasal cannula of claim 11, wherein the first connector inhale port extends from one side of the central hub and is configured to be removably attached to a portion of the first cheek pad.

13. The nasal cannula of claim 12, further comprising a second connector inhale port for attaching to another end of the supply tube.

14. The nasal cannula of claim 13, wherein the second connector inhale port extends from an opposite side of the central hub and is configured to be removably attached to a portion of the second cheek pad.

15. The nasal cannula of claim 9, wherein the central hub includes a valve housing.

16. The nasal cannula of claim 15, wherein the valve housing allows the gas to pass through the inflow passageways.

17. The nasal cannula of claim 15, wherein the valve housing allows the exhalation to pass through the outflow passageways.
